(19) United States
(12) Patent Application Publication
(10) Pub. No.: US 2012/0237929 A1
(43) Pub. Date: Sep. 20, 2012

(54) LONG INTERSPERSED NUCLEAR ELEMENTS (LINE-1) AND ALU HYPOMETHYLATION AS BIOMARKERS FOR COLORECTAL CANCER METASTASIS

(75) Inventors: Ajay Goel, Dallas, TX (US); C. Richard Boland, Dallas, TX (US); Keun Hur, Dallas, TX (US)

(73) Assignee: BAYLOR RESEARCH INSTITUTE, Dallas, TX (US)

(21) Appl. No.: 13/408,947

(22) Filed: Feb. 29, 2012

Related U.S. Application Data
(60) Provisional application No. 61/454,119, filed on Mar. 18, 2011.

(51) Int. Cl. C12Q 1/68; C07H 21/04

(52) U.S. Cl. 435/6.11; 536/23.5

(57) ABSTRACT

A method for determining a colorectal cancer metastasis in a human subject suffering from a primary colorectal cancer (CRC) is described herein. The method of the present invention comprises the steps of: i) identifying the human subject suffering from the primary CRC, ii) obtaining one or more biological samples from the human subject, iii) detecting a methylation level of Alu, LINE-1, or both in the one or more biological samples, and iv) increasing the level of the colorectal metastatic stage in the human subject when the methylation level of Alu, LINE-1 is lower compared to a corresponding control methylation level of Alu, LINE-1.

### STATEMENT OF FEDERALLY FUNDED RESEARCH

This invention was made with U.S. Government support under Contract Nos. R01 CA72851 and CA129286 awarded by the National Cancer Institute (NCI)/National Institutes of Health (NIH). The government has certain rights in this invention.

### CROSS-REFERENCE TO RELATED APPLICATIONS

None.

### TECHNICAL FIELD OF THE INVENTION

The present invention relates in general to the field of cancer detection, prognosis and treatment, and more particularly, to methods for detecting primary colorectal cancers (CRCs) metastasis based on hypomethylation of Alu and LINE-1.

INCORPORATION-BY-REFERENCE OF MATERIALS FILED ON COMPACT DISC

None.

### REFERENCE TO A SEQUENCE LISTING

None.

### BACKGROUND OF THE INVENTION

Without limiting the scope of the invention, its background is described in connection with genetic markers for liver metastasis one of the endpoints of poor prognosis in primary colorectal cancers.

U.S. Patent Application No. 20110039272 (Cowens et al. 2011) discloses a method of predicting clinical outcome in a subject diagnosed with colorectal cancer comprising determining evidence of the expression of one or more predictive RNA transcripts or their expression products in a biological sample of cancer cells obtained from the subject.

U.S. Pat. No. 7,871,769 issued to Baker et al. (2011) provides sets of genes the expression of which is important in the prognosis of cancer. In particular, the invention provides gene expression information useful for predicting whether cancer patients are likely to have a beneficial treatment response to chemotherapy FHIT; MTA1; ErbB4; FUS; BBC3; IGF1R; CD9; TP53BP1; MUC1; IGFBP5; rhoC; RALBP1; STAT3; ERK1; SGCB; DHPS; MGMT; CRIP2; ErbB3; RAP1GDS1; CCND1; PRKCD; Hepsin; AK055699; ZNF38; SEMA3F; COL1A1; BAG1; AKT1; COL1A2; Wnt.5a; PTPD1; RAB6C; GSTM1, BCL2, ESR1; or the corresponding expression product, is determined, said report includes a prediction that said subject has a decreased likelihood of response to chemotherapy.

### SUMMARY OF THE INVENTION

The present invention provides a method for colorectal cancer metastasis in a human subject suffering from a primary colorectal cancer (CRC) comprising the steps of: identifying the human subject suffering from the primary CRC; obtaining one or more biological samples from the human subject; detecting a methylation level of Alu, LINE-1, or both in the one or more biological samples; and increasing the level of the colorectal metastatic stage in the human subject when the methylation level of Alu, LINE-1 is lower compared to a corresponding control methylation level of Alu, LINE-1.

The present invention also provides a biomarker for detecting a colorectal liver metastasis stage in a human subject suffering from primary colorectal cancer comprising a methylation level of Alu, LINE-1, or both for comparison to a corresponding control methylation level of Alu, LINE-1, or both, wherein a lower Alu, a lower LINE-1 methylation or both are indicative of colorectal liver metastatic stage in the human subject.

The present invention provides a kit for determining colorectal liver metastasis including one or more biomarkers to determine a methylation level of Alu, LINE-1, or both and instructions for their use in diagnosing a presence or a risk for colorectal cancer metastasis, instructions for their use in diagnosing the presence or the risk for colorectal cancer metastasis, wherein the instructions comprise providing step-by-step instructions for comparing the methylation level of Alu, LINE-1, or both in one or more samples from a subject suffering from colorectal cancer to a corresponding control methylation level of Alu, LINE-1, or both in one or more samples obtained from a normal subject, wherein the normal subject is a subject not suffering from metastatic colorectal cancer, wherein a lower Alu, lower LINE-1 methylation or both are indicative of colorectal liver metastasis in the human subject.

The present invention also provides a method for selecting a cancer therapy for a patient diagnosed with metastatic colorectal cancer by determining an overall methylation level of Alu, LINE-1, or both in one or more cells obtained from a biological samples of the subject, wherein an Alu methylation level and an LINE-1 methylation level are lower compared to the corresponding control methylation level is indicative of colorectal liver metastasis; and selecting the cancer therapy based on the determination of the stage or presence of the colorectal liver metastasis in the patient.

The present invention also provides a method of performing a clinical trial to evaluate a candidate drug believed to be useful in treating colorectal liver metastasis by a) determining a stage of the metastatic colorectal cancer by a method comprising the steps of: determining an overall methylation level of Alu, LINE-1, or both in one or more cells obtained from a biological samples of the subject, wherein Alu methylation and LINE-1 methylation in liver metastatis were significantly lower compared to the corresponding matched primary CRC is indicative of a stage of the colorectal liver metastasis; b) administering a candidate drug to a first subset of the patients, and a placebo to a second subset of the patients; c) repeating step a) after the administration of the candidate drug or the placebo; and d) monitoring a change in the liver metastases that is statistically significant as compared to any reduction occurring in the second subset of patients, wherein a statistically significant reduction indicates that the candidate drug is useful in treating said disease state.

### BRIEF DESCRIPTION OF THE DRAWINGS

None.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “colorectal cancer” includes the well-accepted medical definition that defines colorectal cancer as a medical condition characterized by cancer of cells of the intestinal tract below the small intestine (i.e., the large intestine (colon), including the cecum, ascending colon, transverse colon, descending colon, sigmoid colon, and rectum). Additionally, as used herein, the term “colorectal cancer” also further includes medical conditions, which are characterized by cancer of cells of the duodenum and small intestine (jejunum and ileum).

As used herein, the term “colorectal metastatic stage” is used to describe the extent to which a colorectal cell with metastatic potential becomes metastatic. For example, in a human patient colorectal cancer cells that have become metastatic exit from the colorectal tissue and metastasize to new metastatic foci, e.g., in the liver. Thus, the various stages of colorectal metastasis as used herein match the extent to which the colorectal cancer cells advance from a benign to an aggressive malignant phenotype typical of more advanced stages of cancer.

The term “tissue sample” (the term “tissue” is used interchangeably with the term “tissue sample”) should be understood to include any material composed of one or more cells, either individual or in complex with any matrix or in association with any chemical. The definition shall include any biological or organic material and any cellular subportion, product or by-product thereof. The definition of “tissue sample” should be understood to include without limitation sperm, eggs, embryos and blood components. Also included within the definition of “tissue” for purposes of this invention are certain defined acellular structures such as dermal layers of skin that have a cellular origin but are no longer characterized as cellular. The term “stool” as used herein is a clinical term that refers to feces excreted by humans.

The term “gene” as used herein refers to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated. The term “allele” or “allelic form” refers to an alternative version of a gene encoding the same functional protein but containing differences in nucleotide sequence relative to another version of the same gene.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., a-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “biomarker” as used herein in various embodiments refers to a specific biochemical in the body that has a particular molecular feature to make it useful for diagnosing and measuring the progress of disease or the effects of treatment. For example, common metabolites or biomarkers found in a person's breath, and the respective diagnostic condition of the person providing such metabolite include, but are not limited to, acetaldehyde (source: ethanol, X-threonine; diagnosis: intoxication), acetone (source: acetoacetate; diagnosis: diet/diabetes), ammonia (source: deamination of amino acids; diagnosis: uremia and liver disease), CO (carbon monoxide) (source: CH2C12 , elevated % COHb; diagnosis: indoor air pollution), chloroform (source: halogenated compounds), dichlorobenzene (source: halogenated compounds), diethylamine (source: choline; diagnosis: intestinal bacterial overgrowth), H (hydrogen) (source: intestines; diagnosis: lactose intolerance), isoprene (source: fatty acid; diagnosis: metabolic stress), methanethiol (source: methionine; diagnosis: intestinal bacterial overgrowth), methylethylketone (source: fatty acid; diagnosis: indoor air pollution/diet), O-toluidine (source: carcinoma metabolite;

diagnosis: bronchogenic carcinoma), pentane sulfides and sulfides (source: lipid peroxidation; diagnosis: myocardial infarction), H2S (source: metabolism; diagnosis: periodontal disease/ovulation), MeS (source: metabolism; diagnosis: cirrhosis), and Me2S (source: infection; diagnosis: trench mouth).

As used herein the term “immunohistochemistry (IHC)” also known as “immunocytochemistry (ICC)” when applied to cells refers to a tool in diagnostic pathology, wherein panels of monoclonal antibodies can be used in the differential diagnosis of undifferentiated neoplasms (e.g., to distinguish lymphomas, carcinomas, and sarcomas) to reveal markers specific for certain tumor types and other diseases, to diagnose and phenotype malignant lymphomas and to demonstrate the presence of viral antigens, oncoproteins, hormone receptors, and proliferation-associated nuclear proteins.

The term “statistically significant” differences between the groups studied, relates to condition when using the appropriate statistical analysis (e.g. Chi-square test, t-test) the probability of the groups being the same is less than 5%, e.g. p<0.05. In other words, the probability of obtaining the same results on a completely random basis is less than 5 out of 100 attempts.

The term “kit” or “testing kit” denotes combinations of reagents and adjuvants required for an analysis. Although a test kit consists in most cases of several units, one-piece analysis elements are also available, which must likewise be regarded as testing kits.

Global hypomethylation of cytosines within CpG dinucleotides is one of the distinguishing features of the neoplastic cells in human cancers. More specifically, hypomethylation of evolutionarily conserved repetitive elements (e.g., Alu and LINE-1) is associated with increased chromosomal instability in colorectal cancer. The present inventors recognized from recent data that transcription start sites of certain proto-oncogenes are located within these repeat elements, and increased hypomethylation of these regions induces the expression of illegitimate oncogenic transcripts. The present inventors recognized that frequent global Alu and LINE-1 hypomethylation of these repeat sequences can be associated with a metastatic phenotype.

In one embodiment, the present invention provides a method of diagnosing and treating colorectal cancers by reviewing the increased hypomethylation of Alu and LINE-1 sequences as an indication of colorectal cancers metastasis development. The present inventors analyzed a panel of colorectal cancers cells with different metastatic potential, as well as tissues from 50 colorectal cancers patients with matched primary colon cancer and corresponding liver metastasis tissues. Alu and LINE-1 methylation levels were determined by quantitative bisulfite pyrosequencing. Lower levels of Alu and LINE-1 methylation were observed in colorectal cancers cell lines that came from metastatic foci.

In clinical specimens Alu and LINE-1 methylation levels showed that Alu methylation in liver metastasis were significantly lower compared to the matched primary colorectal cancers tissues (77.2%±8.3 and 80.9%±10.7, respectively; P<0.01). Similarly, the levels of LINE-1 methylation in metastasized liver foci was significantly lower compared to the corresponding matched primary colorectal cancers (61.2%±9.7 vs 65.8%±7.0 (P<0.01). The relative demethylation level was higher in LINE-1 (4.6%) than Alu (3.7%) sequences. In further support of our results, methylation analysis of surrounding normal liver tissue showed higher methylation levels for Alu (82.2%±4.7) and LINE1 (71.3%±8.3) compared with both primary colorectal cancers and liver metastasis.

By way of example and not a limitation of the present invention, the present inventors illustrated that increased Alu and LINE-1 hypomethylation is a novel feature of liver metastasis from colorectal cancers and the hypomethylation of these repeat elements inadvertently permits activation of previously silenced proto-oncogenes, which may facilitate a more aggressive malignant phenotype in these advanced stage cancers.

It will be understood by one skilled in the art that changes in Alu and LINE-1 hypomethylation could be used as possible markers for metastatic stage discrimination (namely between Stage III and Stage IV metastatic colorectal cancers).

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,871,769: Gene Expression Markers for Predicting Response to Chemotherapy.

U.S. Patent Application No. 20110039272: Gene Expression Markers for Colorectal Cancer Prognosis.

What is claimed is:

1. A method for determining a colorectal cancer metastasis in a human subject comprising the steps of:
obtaining one or more biological samples from the human subject;
detecting a methylation level of Alu, LINE-1, or both in the one or more biological samples; and
increasing the diagnosis of the level of the colorectal metastatic stage in the human subject when the methylation level of Alu, LINE-1 is lower compared to a corresponding control methylation level of Alu, LINE-1.

2. The method of claim 1, wherein the biological samples are selected from the group consisting of a cancer biopsy, a tissue sample, a liver biopsy, a fecal sample, a cell homogenate, a blood, one or more biological fluids, or any combinations thereof.

3. The method of claim 1, wherein the level of methylation was lower in LINE-1 than Alu.

4. The method of claim 1, wherein the methylation level of Alu is indicative of stage II liver metastasis or stage III distant metastasis.

5. The method of claim 1, wherein the methylation levels were determined by quantitative bisulfate pyrosequencing.

6. The method of claim 1, wherein the methylation levels are determined at portions of the LINE-1 than Alu that comprise proto-oncogene start sites.

7. A biomarker for detecting a colorectal cancer metastasis in a human subject suffering from primary colorectal cancer comprising:
a methylation level of Alu, LINE-1, or both for comparison to a corresponding control methylation level of Alu, LINE-1, or both, wherein a lower Alu, a lower LINE-1 methylation or both are indicative of a higher colorectal metastatic stage in the human subject.

8. The biomarker of claim 7, wherein the presence of a lower Alu methylation, a lower LINE-1 methylation or both are indicative of stage II liver metastasis or stage III distant metastasis.

9. A kit for determining a stage of colorectal metastasis comprising:
one or more biomarker to determine a methylation level of Alu, LINE-1, or both; and
instructions for their use in diagnosing presence or risk for colorectal cancer metastasis, wherein the instructions comprise providing step-by-step instructions for comparing the methylation level of Alu, LINE-1, or both in one or more samples from a subject suffering from colorectal cancer to a corresponding control methylation level of Alu, LINE-1, or both in one or more samples obtained from a normal subject, wherein the normal subject is a subject not suffering from metastatic colorectal cancer, wherein a lower Alu, lower LINE-1 methylation or both are indicative of colorectal liver metastatic stage in the human subject.

10. The kit of claim 9, wherein the methylation level of methylation of Alu, LINE-1, or both is indicative metastasis.

11. The kit of claim 9, wherein the methylation level of methylation of Alu, LINE-1, or both is indicative liver metastasis.

12. The kit of claim 9, wherein the methylation level of methylation of Alu, LINE-1, or both is indicative of stage II liver metastasis or stage III metastasis.

13. The kit of claim 9, wherein the one or more samples are selected from the group consisting of a tissue sample, a fecal sample, a cell homogenate, one or more biological fluids, or any combinations thereof

14. A method for selecting a cancer therapy for a patient diagnosed with metastatic colorectal cancer comprising the steps of:
determining an overall methylation level of Alu, LINE-1, or both in one or more cells obtained from a biological sample of the subject, wherein an Alu methylation level and an LINE-1 methylation level are lower compared to the corresponding control methylation level is indicative of an advancement in the stage of the colorectal cancer from a lower to a higher stage; and
selecting the cancer therapy based on the determination of the increase in the stage of the colorectal cancer in the patient.

15. The method of claim 14, wherein the biological samples are selected from the group consisting of a cancer biopsy, a tissue sample, a liver biopsy, a fecal sample, a cell homogenate, a blood sample, one or more biological fluids, or any combinations thereof.

16. The method of claim 14, wherein the level of methylation was lower in LINE-1 than Alu.

17. The method of claim 14, wherein the methylation level of Alu is indicative of stage II liver metastasis or stage III distant metastasis.

18. The method of claim 14, wherein the methylation levels were determined by quantitative bisulfite pyrosequencing.

19. The method of claim 14, wherein the methylation levels are determined at portions of the LINE-1 than Alu that comprise proto-oncogene start sites.

20. A method of performing a clinical trial to evaluate a candidate drug believed to be useful in treating colorectal liver metastasis, the method comprising:
a) determining a stage of the metastatic colorectal cancer by a method comprising the steps of: determining an overall methylation level of Alu, LINE-1, or both in one or more cells obtained from a biological samples of the subject, wherein Alu methylation and LINE-1 methylation in liver metastatis were significantly lower compared to the corresponding matched primary CRC is indicative of a stage of the colorectal liver metastasis;
b) administering a candidate drug to a first subset of the patients, and
a placebo to a second subset of the patients;
a comparable drug to a second subset of the patients; or
a drug combination of the candidate drug and another active agent to a second subset of patients;
c) repeating step a) after the administration of the candidate drug or the placebo, the comparable drug or the drug combination; and
d) monitoring a change in the liver metastases that is statistically significant as compared to any reduction occurring in the second subset of patients, wherein a statistically significant reduction indicates that the candidate drug is useful in treating said disease state.

21. A method for determining a colorectal metastatic stage in a human subject suffering from a primary colorectal cancer (CRC) comprising the steps of:
identifying the human subject suffering from the primary CRC;
obtaining one or more biological samples from the human subject;
detecting a methylation level of Alu, LINE-1, or both in the one or more biological samples; and
increasing the level of the colorectal metastatic stage in the human subject when the methylation level of Alu, LINE-1 is lower compared to a corresponding control methylation level of Alu, LINE-1.

22. The method of claim 21, wherein the biological samples are selected from the group consisting of a cancer biopsy, a tissue sample, a liver biopsy, a fecal sample, a cell homogenate, a blood sample, one or more biological fluids, or any combinations thereof.

23. The method of claim 21, wherein the level of methylation was lower in LINE-1 than Alu.

24. The method of claim 21, wherein the methylation level of Alu is indicative of stage II liver metastasis or stage III distant metastasis.

25. The method of claim 21, wherein the methylation levels were determined by quantitative bisulfite pyrosequencing.

26. The method of claim 21, wherein the methylation levels are determined at portions of the LINE-1 than Alu that comprise proto-oncogene start sites.
